(19) United States
(12) Patent Application Publication
(10) Pub. No.: US 2013/0274778 A1
(43) Pub. Date: Oct. 17, 2013

(54) PATIENT SPECIFIC SURGICAL GUIDE

(71) Applicant: ORTHOSOFT INC., Montreal (CA)

(72) Inventors: Laurence MERCIER, Montreal (CA); Pierre COUTURE, Montreal (CA); Louis-Philippe AMIOT, Montreal (CA)

(73) Assignee: ORTHOSOFT INC., Montreal (CA)

(21) Appl. No.: 13/863,678

(22) Filed: Apr. 16, 2013

Related U.S. Application Data
(60) Provisional application No. 61/624,593, filed on Apr. 16, 2012.

(51) Int. Cl. A61B 17/17; G06T 7/00; A61B 19/00

(52) U.S. Cl. CPC A61B 17/1739; A61B 19/50; G06T 7/0012
USPC 606/172; 382/131

(57) ABSTRACT

A patient specific instrument (PSI) surgical guide and method for producing same is described. The method includes: obtaining imagery of at least a portion of a patient, and determining one or more surgical targets in the tissue; planning at least a trajectory of the surgical procedure based on a determined surgical target within the tissue; performing segmentation of the imagery; creating a three-dimensional model of the PSI surgical guide, the PSI surgical guide being customized in size and shape and configured to fit on the specific patient. The PSI surgical guide is then produced to correspond to the modeled PSI surgical guide. The PSI surgical guide has a guide element positioned and oriented to guide a surgical implement along the planned trajectory toward the determined surgical target in the tissue.

### CROSS-REFERENCE TO RELATED APPLICATIONS

The present application claims priority on U.S. Patent Application No. 61/624,593 filed Apr. 16, 2012, the entire content of which is incorporated herein by reference.

### TECHNICAL FIELD

The present application relates generally to surgical instruments and tools for use with computer assisted surgery systems, and more particularly to patient specific instruments and surgical tools for insertion and/or removal of material, including but not limited to instruments for use in cranial applications such as those for conducting brain tissue biopsies.

### BACKGROUND

Surgical instruments and the associated techniques for removing a tissue sample from a surgical site of a patient, such as conducting tissue biopsies for example, often include a needle, syringe or other withdrawal instrument which is positioned into place by the surgeon such that the tissue sample is collected from the desired, targeted, area of the patient's tissue.

One particular application where the withdrawal of tissue sample is particularly difficult, and which requires very precise accuracy, is for cranial applications wherein brain tissue biopsies are to be performed, for example, in order to evaluated and characterize a brain tumor.

When patients present with symptoms that could be associated with a brain tumor, they generally first get a computed tomography (CT) scan. If the CT scan shows a lesion, magnetic resonance imaging (MRI) will generally be ordered because it provides better soft tissue contrast. Surgeons can often broadly categorize a tumor by looking at the MRI results. Because the course of treatment is not the same for all brain tumors, if a doubt remains about the tumor type, surgeons generally perform a biopsy of the brain tissue at the suspected tumor site.

A biopsy consists of taking a tumor sample to examine it under the microscope. There are three main techniques commonly employed to open the skin and bone of the cranium to take a brain biopsy. Firstly, a surgeon can drill directly through the skin and bone, in order to permit access to insert a needle into a desired location of the brain for the removal of the tissue. Secondly, the surgeon can first perform a skin incision, following which a small burr hole is drilled in the skull. A needle is then inserted through the burr hole, into the brain, to proceed with the removal of the tissue. Thirdly, a classical craniotomy can be performed, wherein a bone flap is temporarily removed from the skull to permit a larger access the brain.

In all of the above cases, the surgeon will typically use an image obtained from the MRI or CT scan of the brain, in order to plan the precise location for bone removal and the appropriate angle of access to the relevant brain tissue areas. The amount of skull that needs to be removed depends to a large extent on the type of surgery being performed. The removed bone may then be replaced using titanium plates and screws or another form of fixation (wire, suture, . . . etc).

Many brain biopsies performed today are performed with the additional guidance of a computer assisted surgical navigation system, which may include a frameless system which doesn't require the use of stereotactic frames. Such stereotactic frames are less practical as they require the head frame to be bolted onto the skull of the patient, making the process more complicated and time consuming. Regardless, from the moment the patient is anesthetized and clamped, even a navigation-guided biopsy takes at least 20-30 minutes, but more typically averages about 1 hour, and in worst cases can require up to 2-3 hours.

Computer navigated guided frameless biopsies can be relatively long procedures, particularly when deep seated tumors are involved, because minor rotational errors in the patient registration can lead to a significant movement/navigation error in the deep-seated tissues. Surgeons can also potentially miss their target in such cases and may need to re-position their needle multiple times. Furthermore, the neuronavigation systems which are used are expensive and bulky in the operating room.

### SUMMARY

Therefore, in accordance with one aspect of the present disclosure, there is provided a method of producing a patient specific surgical guide helmet for guiding a cranial surgical procedure adapted to be performed on the head of a patient, comprising the steps of: a) obtaining imagery of at least a portion of the head of the patient, and determining one or more surgical targets in at least one of the brain tissue, the cranium bone and the scalp; b) planning at least a trajectory of the surgical procedure based on the determined surgical target; c) performing segmentation of said imagery; d) creating a three-dimensional model of the patient specific surgical guide helmet using the results of steps a), b) and c), the patient specific surgical guide helmet being customized in size and shape and configured to fit on the scalp of the specific patient, the patient specific surgical guide helmet having at least one guide element incorporated therewith, the guide element being positioned and oriented to guide a surgical implement along the planned trajectory toward the determined surgical target; and e) producing the patient specific surgical guide helmet to correspond to the modeled patient specific surgical guide helmet of step d).

There is also provided, in accordance with another aspect of the present disclosure, a patient specific surgical guide helmet for guiding a cranial surgical procedure to be performed on the head of a patient, comprising: a head-covering portion customized in size and shape to fit directly onto at least a portion of the head of the specific patient, the head-covering portion including at least a first component extending in the sagittal plane and at least a second component extending in the medial-lateral plane, such as to secure the head-covering portion to the head; and at least one guide element integrated into the head-covering portion, the guide element being positioned and oriented to guide a surgical implement along a planned trajectory to reach a predetermined surgical target of the cranial surgical procedure.

There is also provided, in accordance with another aspect of the present disclosure, a system for creating a patient-specific instrument for cranial surgery, comprising: a surgical guide helmet generator for producing a surgical guide helmet model for positioning on the head of a patient; a geometry identifier for identifying a helmet geometry; and a patient specific instrument model generator for creating the patient-specific instrument from said helmet model, the patient specific instrument being adapted to be received on the head of the patient and to guide surgical procedures.

There is further provided, in accordance with another aspect of the present disclosure, a method of performing a cranial surgical procedure on the head of a patient, comprising the steps of: imaging at least a selected portion of the head of the patient and determining one or more surgical targets in at least brain tissue of the patient; planning at least one of a trajectory, cranium entry point and end point of the surgical procedure based on the determined surgical targets; forming a patient specific surgical guide helmet customized to the head of the specific patient based on said imaging and said planned surgical procedure, the patient specific surgical guide helmet having at least one guide element which guides a surgical implement during the cranial surgical procedure along the planned trajectory, the guide element being positioned and oriented such that an axis thereof corresponds to the planned trajectory; securing the patient specific surgical guide helmet onto the head of the patient; and performing the cranial surgical procedure using the guide element of the patient specific surgical guide helmet to guide the surgical implement along said axis through said planned trajectory to the determined surgical target in the brain tissue of the patient.

There is further provided, in accordance with another aspect, a method of performing a surgical procedure involving injecting or removing material from a target location in tissue of a patient, the method comprising the steps of: imaging at least said tissue of the patient to generate a virtual model of the tissue, and determining one or more target locations within the virtual model of the tissue; forming a patient specific surgical guide, customized to fit on the specific patient over a site comprising the tissue and for guiding a surgical implement used to perform the surgical procedure, by: obtaining the imagery of the tissue of the patient; planning at least one of a trajectory and end point of the surgical procedure based on the determined target location; performing a segmentation of the virtual model of the tissue; modeling the patient specific surgical guide to fit on the patient and having at least one guide element incorporated therein, the guide element including at least one aperture providing access to the tissue and defining an entry point for the surgical implement, the guide element being positioned and oriented to guide the surgical implement along the planned trajectory to the determined target location; and creating the patient specific surgical guide which corresponds to the modeled patient specific surgical guide; securing the patient specific surgical guide onto the patient; and performing the surgical procedure using the patient specific surgical guide to guide the surgical implement to the determined target location in the tissue of the patient, including performing at least one of an injection of material into the target location and a removal of tissue from the target location.

There is further provided, in accordance with another aspect, a patient specific surgical guide for guiding a surgical procedure to be performed at a predetermined target location in tissue of a patient, comprising: a patient-specific base portion customized in size and shape to fit directly onto a body part the specific patient, over a site comprising said tissue; and at least one guide element integrated into the base portion, the guide element being positioned and oriented to guide a surgical implement along a planned trajectory to reach the predetermined target location within the tissue, the guide element including at least one aperture providing access to the body part and defining an entry point for the surgical procedure, and the guide element including a guide tube extending outwardly from the aperture and defining an axis centrally therein which extends through the guide tube in alignment with the aperture, the axis corresponding to the planned trajectory.

### BRIEF DESCRIPTION OF THE DRAWINGS

FIG. 1 is a side perspective view of a patient specific surgical guide helmet in accordance with an embodiment of the present disclosure.

FIG. 2 is a front view of the patient specific surgical guide helmet of FIG. 1.

FIG. 3 is a block diagram of a manufacturing process of the patient specific surgical guide helmet of FIG. 1.

FIG. 4 is a block diagram of a patient specific instrumentation computer-assisted surgery system for cranial surgery in accordance with the present disclosure.

FIG. 5 is a schematic side view of a patient specific surgical guide in accordance with an alternate embodiment of the present disclosure.

### DETAILED DESCRIPTION

The present disclosure describes a patient specific instrument (PSI) which provides a surgical guide for performing a surgical procedure at a predetermined target location in tissue of a patient. The term “tissue” as used herein is intended to include both soft tissue and hard tissue (such as bone). Such a surgical procedure may include, but is not limited to, cranial applications such as brain biopsies for example. In all cases, the surgical guide as described herein is customized for each specific patient and is thus said to be a “patient specific instrument” (PSI). The presently described patient specific surgical guide permits such surgical procedures to be performed in a rapid and accurate manner. The surgical procedures capable of being carried out by the PSI surgical guides as described herein may involve injecting material into, and/or removing material from, one or more target locations in the tissue of the patient. While in at least one embodiment described herein, the presently described PSI surgical guide will be particularly described with respect to its use as a head PSI which configured to enable a tissue biopsy or other cranial surgery to be performed on the head of a patient, the patient specific surgical guide of the present invention may be used in other applications, for example for the injection of organic or non-organic material into a bone or soft-tissue site, or the remove of material (for biopsy purposes or otherwise) from either soft tissue or hard tissue.

Further, while in at least one embodiment the PSI surgical guide as described herein may be used without a navigation system, thereby permitting the reduction of overall costs for the procedure. However, in an alternate embodiment, the present PSI surgical guide may also be used in conjunction with a computer assisted surgery (CAS) guidance system for more complex procedures, whereby at least one trackable element (such as an optical tracker or an electronic micro-electromechanical sensor (MEMS) which may include accelerometers and/or gyroscopes for example) is affixed to the head PSI 10. Such trackable elements are disposed in communication with the CAS system with which they are employed such that the CAS system is then able to locate and track (i.e. navigate) the head PSI to which the trackable element is fastened.

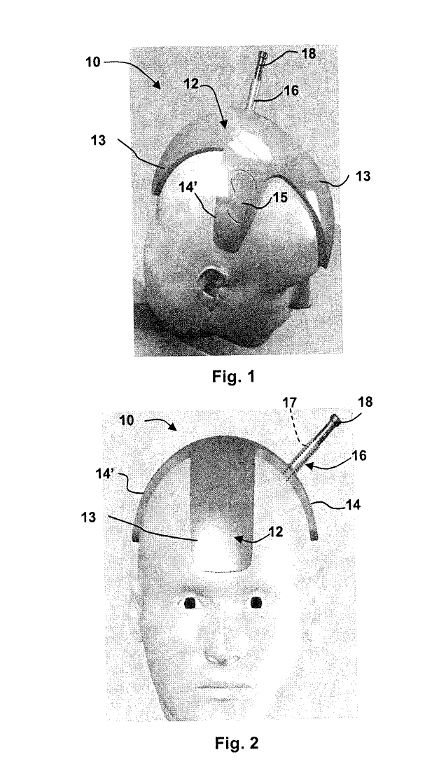

The PSI surgical guide will now be described, referring to FIGS. 1-4, with respect to an embodiment thereof which is particularly intended for use with conducting a cranial surgical procedure, and thus may be generally referred to a “head PSI”. However, as noted above, such a system, the PSI guide itself, as well as its method of use and the method of producing same may also be useful for use in other, non-cranial or non-head, applications, for both the withdrawal of tissue (such as for conducting biopsies of other patient bone and/or soft tissue sites) and/or for the injection of material (whether such material is patent tissue or non-organic materials such as bone cement, etc).

Referring now to FIG. 1, a patient specific surgical guide helmet 10 is provided. The surgical guide helmet 10 described herein, once placed on a patient's head, will guide the surgeon during biopsy and/or during incision. The patient specific instrument (PSI) and associated CAS system, including the surgical guide helmet 10, will ensure that due to the accuracy of the fit of the helmet 10 on the head of the patient in a repeatable and unique position and the configuration thereof in view of the predetermined cranial application to be performed, a patient specific surgical procedure on the head is rendered possible. Once placed in position on the patient's head, the surgical guide helmet 10 will not interfere with any head clamps which may be used during the cranial procedure, such as a Mayfield™ clamp for example, and will impose a movement constraint to the patient head's. A different surgical guide helmet 10 is accordingly produced for each patient, such that the surgical guide helmet 10 is a patient specific instrument designed and produced expressly and uniquely for each patient.

As seen in the embodiment of FIGS. 1-2, the surgical guide helmet 10 includes a curved head-covering portion 12 which is molded or otherwise shaped, as will be seen, to fit directly onto the scalp of the specific patient's head. The curved head-covering portion 12 according covers at least a portion of the patient's scalp and has an inwardly facing surface that is formed such that it corresponds exactly to the shape and contours of the head of the patient to be operated on. The curved head-covering portion 12 is therefore purpose built and shaped exclusively such as to fit onto the head of the specific patient for which it is intended. The curved head-covering portion 12 may be shaped like a cross, comprising a sagittal C-shape component 13 which extends for-aft in the sagittal plane and transversely extending side wings 14 and 14′ which extend in the medial-lateral plane that is perpendicularly disposed relative to the sagittal plane. The side wings 14 and 14′ can be manufactured separately from the sagittal C-shape component 12, for example to permit a smaller disassembled envelope in order to simplify transport and storage, in which case the side wings 14, 14′ may simply clip onto the sagittal C-shape component 12 in a precise position relative thereto, once assembled prior to installation of the surgical guide helmet 10 prior to performing the surgical procedure. Alternately, the various portions of the curved head-covering portion may be each separately formed and subsequently fastened together to create the finished surgical guide helmet 10 prior to installation on a patient's head. In another alternate embodiment, for applications in which tumors are located within the brain tissue at a position which is far from the shape of the head covering portion 12 (which in the depicted embodiment has a generally X-shaped configuration), it may also be possible to have alternately shaped head-covering portions and/or to have additional components thereof which may be “clipable” or otherwise attached to the basic shape of the head-covering portion 12 of the head PSI 10, in order to cover a larger portion of the patient's scalp.

The surgical guide helmet 10 includes at least one entry point for the insertion therethrough of a cutting tool, such as a drill bit or burring tool for example, as well as the insertion of a biopsy needle or other surgical tool. This entry point comprises, in at least the depicted embodiment, a biopsy guide tube 16 which extends radially outwardly from the outer surface of the curved head-covering portion 12, and which defines therethrough a bore 17 extending fully through the thickness of the guide tube 16 and the curved head-covering portion 12 of the surgical guide helmet 10, thereby providing localized access to the skin and underlying bone that is to be resected by the surgeon for the purposes of inserting a biopsy needle or other surgical implement. The specific location and orientation of the guide tube 16 on the head-covering portion 12 is specifically selected in order to correspond to the exact desired biopsy site(s) within the brain for the patient in question.

As such, a drill bit or other drilling tool, and subsequently a biopsy needle, can be inserted through the bore 17 which extends through the guide tube 16 of the surgical guide helmet 10. Given that the guide tube 16 has been formed on the head-covering portion 12 of the surgical guide helmet 10 in a position and orientation which corresponds exactly to a desired tumor or brain tissue sampling site, the guide tube 16 permits a cutting tool, biopsy needle or other surgical implement to be quickly and easily introduced into the bone or brain tissue in a precise position required given the particular anatomical conditions of the specific patient in question.

In an alternate embodiment, multiple entry points, such as guide tubes 16 or otherwise, may also be provided on the surgical guide helmet 10, so has to allow the surgeon to access multiple tumors or to access one tumor with multiple biopsy needles, inserted at different points and/or at different angles through the skull. Thus, the surgical guide helmet 10 described herein provides an effective device for a surgeon to plan multiple biopsy targets and provides some flexibility around a chosen target to access to the target.

In another embodiment, where the tumor is a superficial tumor and a skin incision is sufficient to allow a biopsy procedure or even the removal of the tumor, a tracing guide 15 can also be designed on the surgical guide helmet 10 (see FIG. 1), either in addition to or in lieu of the guide tube 16. The tracing guide 15 may constitute another type of entry point, and is similarly used by the surgeon as a patient specific cutting guide such as to quickly and accurately perform the required cranial application or surgical step. The tracing guide 15 will act as a precision tool during the incision of the skin and skull, for example to expose the brain and provide access to the tumor.

As seen in FIG. 2, a biopsy depth guide or depth gauge 18 may be provided within the guide tube 16, so as to control the depths of entry of a biopsy needle or other surgical implement inserted therethrough. The biopsy depth guide 18 comprises a hollow tube which slidingly fits within the guide tube 16 such as to be displaceable inwardly and outwardly within the outer tube 16. The biopsy guide 18 may have markings thereon such as to provide a visual depth gauge which indicates to the surgeon how deep into the skull and/or brain tissue the biopsy needle or other implement has been inserted, and may also be adjusted prior to surgery so as to provide a limit or control stop to prevent a maximum desired depth from being reached. Therefore, not only does the surgical guide helmet 10 permit the trajectory of the needle by the positioning and orientation of the guide tube 16 at the entry point, but the depth of the insertion can also be monitored and controlled by the biopsy guide 18. It is also to be understood that the surgeon may plan a biopsy which is to be performed at more than one depth, while still using the same biopsy guide tube 16. For example, the sliding depth gauge 18 may be pre-marked with two (or more) different desired target depths for the biopsy, such as to visually indicate to the surgeon the depth of the needle at the two desired depths.

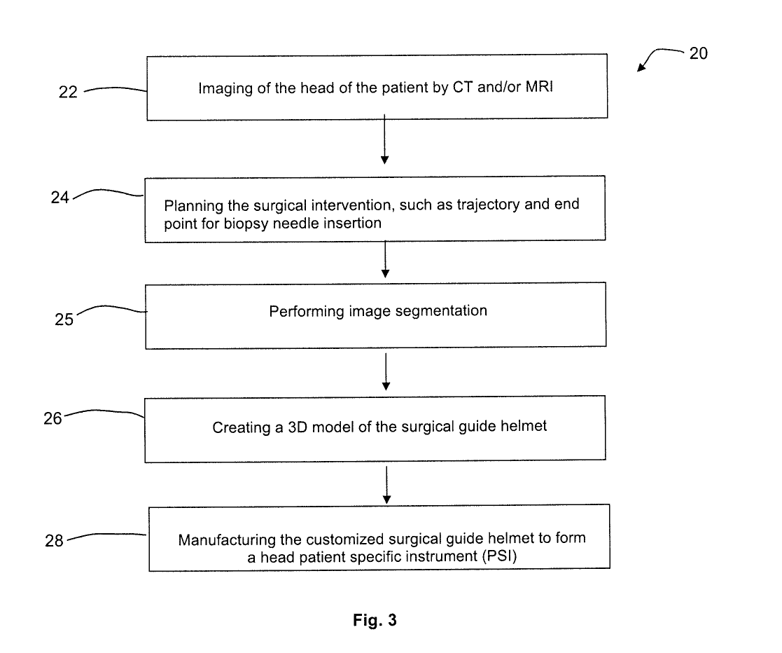

Referring now to FIG. 3, a method 20 of manufacturing and/or using the patient specific surgical guide helmet 10 is generally described. Generally, the surgical guide helmet 10 is designed for the specific patient and produced pre-surgery, such as to provide a custom, patient specific surgical guide 10 which, once installed in place on the patient, will allow the surgeon to rapidly and accurately performed the planned cranial surgical procedure (such as a brain tissue biopsy, for example), without the need for guidance by a CAS neuronavigation system. In the initial step 22 of the method 20, preparation steps for 3D planning of the surgical guide helmet 10 are accomplished by first imaging the head of the patient using a CT and/or MRI scan, pre-operatively.

From the data obtained in this step 22, the surgeon then plans and selects, in step 24, the desired cranial surgical procedure to be performed, based on the imaging results. This planning may include, for example, selecting the trajectory and entry and end points for a biopsy of a brain tumor. This step 24 may be accomplished by the surgeon using a CAS neuronavigation system, but such a navigation need not necessarily be used. At least one of a trajectory and an end point of the surgical procedure is planned during this step, based and depending on the determined surgical target. These steps may vary slightly depending on whether the surgical guide helmet 10 is used for a biopsy or for the resection of a brain tumor. In the case of a tumor resection, there remains a planned trajectory, however the tumor becomes a targeted region, more than a single end point. For biopsies, and more precise end point may be determined and targeted. Several alternate biopsy targets, entry points and/or trajectories may also be selected by the surgeon. This step may include, for example, determining the preferred position for the entry point of the drill and/or biopsy needle, determining the orientation of the required trajectory axis for the insertion thereof such as to reach the tumor site, and the end point along this axis within the brain. The input of the surgeon at this step therefore allows the position of the tumor or surgical target site within the brain tissue to be accurately determined, based on the imaging results.

Once the planning step 24 has been completed, and prior to any computer model being generated, segmentation step 25 is performed whereby the image results obtained in step 22 are segmented. This may include segmenting the scalp skin or the cranial bone itself, and/or segmentation of the brain tissue and/or a tumor within the brain tissue. Other soft tissue may also be segmented, such as vascular structures, etc. In all cases, this is accomplished on the imagery of these head tissues using a computer system and/or segmentation software. While this computer system can be a CAS navigation system, such an expensive and complex system need not be used for this relatively straightforward image segmentation step. For example, a much cheaper system can be used to perform the image segmentation. In fact, a very simple interface made just for segmentation and planning may be used, which is more cost effective than using a much more complex CAS navigation system.

Regardless, the segmentation of the image is then used to generate the 3D model of the scalp, cranial bone and/or brain tissue, and therefore of the surgical guide helmet 10 to be installed in place on the patient's head.

The information from steps 22, 24 and 25 is then used, in step 26, in order to create a 3D model of the surgical guide helmet 10. This step 26 therefore also includes determining the corresponding position of the guide tube 16 and/or cut tracing guide 15 of the patient specific surgical guide helmet 10, as is required based on the patient's anatomical criteria as determined by the imaging results of step 22 and the planned surgical procedure determined in step 24. Thus, by combining the data obtained in steps 22 and 24 with the segmented image of step 25, a 3D model of the surgical guide helmet 10 is conceived and generated in step 26. The 3D model of the surgical guide helmet 10 so created has the guide tube(s) 16 and/or cut tracing guide(s) 15 in the precise desired locations on the curved head-covering portion 12 of the helmet 10 and the required orientation thereof such as to define desired the determine biopsy trajectory axis, as is required for the anatomical conditions of the specific patient and the surgical cranial procedure to be performed. Several different guides 15, 16 may be provided on the same helmet 10, be they guide tubes 16 for biopsy needle insertion, cut tracing guides 15 for making skin and/or bone cuts, or otherwise. For example, these guides of the helmet 10 may also include a guide for the insertion of deep brain electrodes, in which case several access tubes (similar to the biopsy tube 16 but smaller) providing access to the brain tissue beneath the helmet 10 would be provided through which the electrodes can be inserted. These multiple guides, be they cutting guides or otherwise, may correspond to primary and alternate planned biopsy trajectories, for example, such as to permit the surgeon to quickly perform the alternate planned interventions during surgery, which have been designed pre-operatively during the step 24, without having to plan and devise such alternately surgical steps intraoperatively. In addition to the precise positioning of the cutting guide features 15 and 16, the modeled surgical guide helmet 10 is also shaped such that the curved head-covering portion 12 thereon is formed specifically to fit onto the head of the head of the patient, using the imaging information from the CT and/or MRI scans. The shape and configuration of the curved head-covering portion 12 may also be selected such as to avoid any potential interference with the head clamp (Mayfield clamp, etc.) which is to be used during the surgery to keep the patient's head in a fixed position.

Once the 3D thus model is determined and validated, the surgical guide helmet 10 is formed in step 28, according to this exact 3D model that has been exclusively designed for the specific patient and the cranial surgical procedure to be performed. This forming step 28 may include the manufacture of the patient specific surgical guide helmet 10 using a rapid production technique, such as rapid prototyping, 3D printing, laser deposition, etc. which creates the helmet 10 quickly and accurately based on the digital 3D model thereof created in step 26.

The surgical guide helmet 10, once formed as described above, can then be positioned on the head of the patient and fixed in place thereon, thereby providing a tailor made surgical guide which has been designed and produced exclusively for the patient in question, and which permits the surgeon to rapidly and accurately conduct the desired cranial surgical operation without requiring additional guidance by a neuronavigation CAS system.

The surgical guide helmet 10 therefore constitutes a head component of the overall PSI system, which may also include a “navigation” component. The head component formed by the surgical guide helmet 10 ensures that the PSI is accurately placed on the head of the patient for which it is designed in a repeatable and unique position, and provides precise positional guidance for the cranial surgical procedure to be performed, such as the insertion of a biopsy needle through the guide tube 16, to a desired depth into the brain tissue as guided by the depth gauge 18 or the incision made following the contours of the tracing guide 15 for a superficial tumor for example. The increased precision which is enabled by the present system, as described hereinabove, allows for a patient specific device to be designed and produced for use by a neurosurgeon to conduct a brain tumor biopsy or other cranial surgical intervention more rapidly and repeatably for the specific patient. While the use of the present cranial PSI may, in some cases, permit the use of a neuronavigational CAS system to be limited if not eliminated during the surgery, such as CAS system may still be used as a fall back by the surgeon when used in conjunction with the present PSI 10. It is estimated that the presently described system and PSI 10 will be able to reduce the 20-30 minutes typically required for the navigation portion of a brain tumor biopsy to about 2 minutes when using the PSI described herein.

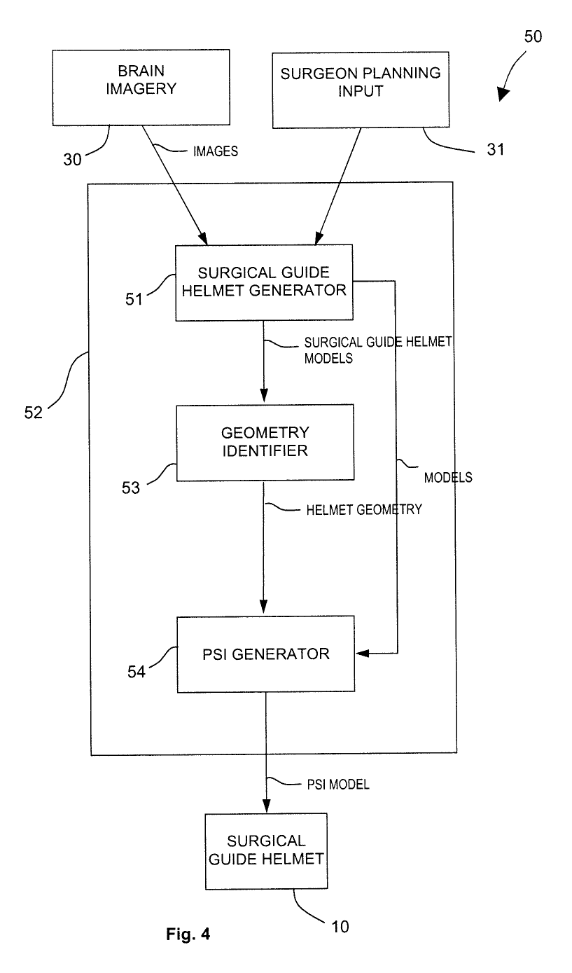

Referring now to FIG. 4, a system for creating and manufacturing the surgical guide helmet 10 is generally shown at 50. The system 50 may be a computer assisted surgery (CAS) system, or a stand alone system for the manufacture of the head PSI 10 as described above. The system 50 receives at least brain imagery 30 from any appropriate imaging technology (e.g., MRI, enhanced-contrast CT, etc.). The imaging technology apparatus may be a part of the system 50, or remote therefrom. The imagery 30 may comprise three-dimensional images of the brain and more particularly of the tumor location and morphology. The system also receives an input 31 which comprises the surgeon's planning for the cranial surgical procedure to be performed using the head PSI 10. This may include, in the case of a brain biopsy for example, the biopsy needle trajectory and exact biopsy location(s).

The system 50 comprises a processor unit 52 that receives the brain tissue images 30 and the surgeon's planning input 31, and that will produce PSI models from this data. The processor unit 52 has a processor to run the software application that will generate the PSI models. Accordingly, the processor unit 52 may be any appropriate computer or processing unit. User interfaces (e.g., monitor, screen, keyboard, mouse, touch-screen) are part of the system 50 in communication with the processor unit 52, for the involvement of an operator in the creation of the PSI models.

The processor unit 52 comprises a surgical guide helmet generator 51. The surgical guide helmet generator 51 is used to interpret the images 30 and surgeon's planning input data 31, and thus to create a 3D model of the surgical guide helmet. The 3D model distinguishes the position of the tumor for example, including not only the location (depth in the brain tissue, orientation, etc.) but also the morphology in order to plan a biopsy. The surgeon's or operator's input 31 may also be required for confirming the proper segmentation of the brain and/or skin tissue which is segmented by the processor unit 52. Typically, manual or semi-automatic segmentation of the tumor is required from the surgeon if the head PSI 10 is being used to navigate a tumor resection. In the case when the surgical procedure is a biopsy, tumor resection may be useful but is not absolutely required. The interfaces may be used for these purposes.

A geometry identifier 53 uses the model of the brain and head of the patient to determine the dimension of the surgical guide helmet. The geometry identifier 53 defines the geometrical parameters of the helmet, such as dimensions, thickness, curvature, position and orientation of the guides 15, 16, etc.

Still referring to FIG. 4, a PSI generator 54 uses the helmet geometry, and brain/head imaging to produce PSI models. The PSI model is used to manufacture the patient-specific surgical guide helmet 10 that will be used during the planed cranial surgery on the specific patient. This final manufacture may include the creation of the patient specific surgical guide helmet 10 using a rapid prototyping or rapid manufacturing process, for example.

The PSI can be used for cranial biopsy, but it can also be adapted to be used for the resection of lesions such as tumors, drainage of cysts, or the insertion of deep brain stimulation electrodes, etc.

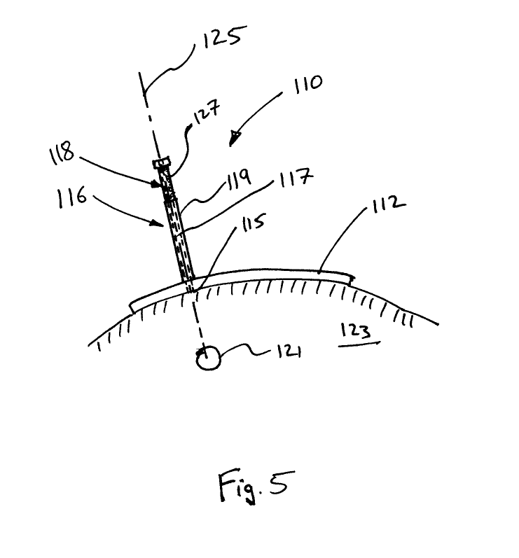

Referring now to FIG. 5, a patient specific (PSI) surgical guide 110 for guiding a surgical procedure to be performed at a predetermined target location 121 in tissue 123 of a patient is shown. The PSI surgical guide 110 include, much as per the PSI guide helmet 10 described above, a patient-specific base portion 112 and at least one guide element 116. The guide element 116 maybe integrally formed with the patient-specific base portion 112, or may be seperately formed and then integrated into the base portion, whether by fastening or otherwise forming the two components together. The patient-specific base portion 112 is customized in size and shape to fit directly and precisely onto the tissue 123 of the body part the specific patient, over a site comprising the tissue 123 and the target location 121 therein. The guide element 115 is positioned and oriented such as to guide a surgical implement along a planned trajectory to reach the predetermined target location 121 within the tissue 123. The guide element 116 includes at least one aperture 115, which may be disposed for example in an inner facing surface of the base portion 112, which aperture 116 provides access to the body part and defines an entry point for the surgical procedure. The guide element 116 includes a guide tube 119 extending outwardly from the aperture 115 and from the base portion 112. The hollow guide tube 119 defines centrally therein an axis 125 which extends through the bore 117 of the guide tube 119 in alignment with the aperture 115. The axis 125 corresponds to the planned trajectory along which the surgical implement is to be fed such as to reach the target location 121 within the tissue 123.

The guide element 116 also includes a depth guide or depth gauge 118 provided within the guide tube 116, so as to control the depths of entry of a needle or other surgical implement inserted therethrough. The depth guide 118 comprises a hollow tube which slidingly fits within the larger guide tube 116 such as to be slidingly displaceable inwardly and outwardly within the outer tube 116. The biopsy guide 18 may have visual markings 127 thereon such as to provide a visual depth gauge which indicates to the surgeon how deep into the tissue the surgical implement has been inserted, and may also be adjusted prior to surgery so as to provide a limit or control stop to prevent a maximum desired depth from being reached. Therefore, the PSI surgical guide 110 permits not only the trajectory of the surgical implement to be fixed by the positioning and orientation of the guide tube 116 at the determined entry point, but also enables the depth of the insertion to be at least monitored if not physically limited and thus controlled.

In a manner similar to that described above, the method of performing a surgical procedure involving injecting or removing material from a target location in tissue of a patient using the PSI surgical guide 110 may include, for example, first imaging patient tissue to generate a virtual model of the tissue and determining one or more target locations within the virtual model of the tissue, and then forming the patient specific surgical guide 110, which is customized to fit on the specific patient over a site comprising the tissue and for guiding a surgical implement used to perform the surgical procedure. This process of forming the PSI surgical guide 110 may include, for example, obtaining the imagery of the tissue, planning at least one of a trajectory and end point of the surgical procedure based on the determined target location, performing a segmentation of the virtual model of the tissue, modeling the patient specific surgical guide to fit on the patient; and then creating the PSI surgical guide corresponding to the modeled patient specific surgical guide. The PSI surgical guide 110 so created has at least one guide element including at least one aperture providing access to the tissue and defining an entry point for the surgical implement, the guide element being positioned and oriented to guide the surgical implement along the planned trajectory to the determined target location. The method may further then include securing the so-formed patient specific surgical guide onto the patient, and performing the predetermined surgical procedure using the patient specific surgical guide to guide the surgical implement to the determined target location in the tissue of the patient, including performing at least one of an injection of material into the target location and a removal of tissue from the target location.

The above description is meant to be exemplary only, and one skilled in the art will recognize that changes may be made to the embodiments described. For example, although the PSI system of the present invention has been generally described above with respect to a PSI 10 used for cranial applications such as biopsies, it is to be understood that the presently described PSI system may also be adapted and used for the drainage of cysts, the resection of superficial brain tumors and for shunts, etc. Still other modifications which fall within the scope of the present invention will be apparent to those skilled in the art, in light of a review of this disclosure, and such modifications are intended to fall within the appended claims.

1. A method of producing a patient specific surgical guide helmet for guiding a cranial surgical procedure adapted to be performed on the head of a patient, comprising the steps of:
a) obtaining imagery of at least a portion of the head of the patient, and determining one or more surgical targets in at least one of the brain tissue, the cranium bone and the scalp;
b) planning at least a trajectory of the surgical procedure based on the determined surgical target;
c) performing segmentation of said imagery;
d) creating a three-dimensional model of the patient specific surgical guide helmet using the results of steps a), b) and c), the patient specific surgical guide helmet being customized in size and shape and configured to fit on the scalp of the specific patient, the patient specific surgical guide helmet having at least one guide element incorporated therewith, the guide element being positioned and oriented to guide a surgical implement along the planned trajectory toward the determined surgical target; and
e) producing the patient specific surgical guide helmet to correspond to the modeled patient specific surgical guide helmet of step d).

2. The method of claim 1, wherein step a) further comprising obtaining imagery of one or more of brain tissue, cranium bone and scalp of the head.

3. The method of claim 2, wherein step c) further comprises performing segmentation of at least the imagery of the scalp upon which the patient specific surgical guide helmet will sit.

4. The method of claim 3, further comprising performing segmentation of the imagery of the brain tissue.

5. The method of claim 1, wherein step d) further comprises creating the guide element of the patient specific surgical guide helmet to have at least one aperture providing access to the cranium at a planned entry point in the scalp and cranium for the surgical procedure, and the guide element defining an axis extending fully therethrough which corresponds to the planned trajectory.

6. The method of claim 5, further comprising forming the guide element to include a guide tube extending radially outwardly from said aperture and defining said axis centrally therein, the guide tube guiding the surgical implement therethrough along the planned trajectory.

7. The method of claim 1, wherein step d) further comprises forming the patient specific surgical guide helmet with a head-covering portion having a sagittal C-shape component extending in the sagittal plane and two transversely extending side wing components extending from the sagittal C-shape component in the medial-lateral plane.

8. The method of claim 1, wherein step b) further comprises planning an end point of the surgical procedure at the determined surgical target.

9. The method of claim 1, further comprising providing the patient specific surgical guide helmet with at least one trackable element thereon, the trackable element being operable for communication with a computer assisted surgery guidance system.

10. A patient specific surgical guide helmet for guiding a cranial surgical procedure to be performed on the head of a patient, comprising:
a head-covering portion customized in size and shape to fit directly onto at least a portion of the head of the specific patient, the head-covering portion including at least a first component extending in the sagittal plane and at least a second component extending in the medial-lateral plane, such as to secure the head-covering portion to the head; and
at least one guide element integrated into the head-covering portion, the guide element being positioned and oriented to guide a surgical implement along a planned trajectory to reach a predetermined surgical target of the cranial surgical procedure.

11. The patient specific surgical guide helmet of claim 10, wherein the head-covering portion forms a cross-shape and includes a sagittal C-shape component extending in the sagittal plane and transversely extending side wing components extending from the sagittal C-shape component in the medial-lateral plane.

12. The patient specific surgical guide helmet of claim 10, wherein the guide element includes at least one aperture providing access to the scalp and/or cranium and defining a planned entry point therein for the surgical procedure, and the guide element defining an axis extending fully therethrough in alignment with the aperture, the axis corresponding to the planned trajectory.

13. The patient specific surgical guide helmet of claim 12, wherein the guide element includes a guide tube extending radially outwardly from said aperture and defining said axis centrally therein, the guide tube guiding the surgical implement therethrough along the planned trajectory.

14. The patient specific surgical guide helmet of claim 13, wherein the guide element includes a depth gauge provided within the guide tube, the depth gauge measuring and controlling depth of the surgical implement inserted through the guide tube.

15. The patient specific surgical guide helmet of claim 14, wherein the depth gauge comprises a hollow tube which slidingly fits within the surrounding guide tube and which includes visual depth markings on an outer surface thereof.

16. The patient specific surgical guide helmet of claim 10, wherein the patient specific surgical guide helmet with at least one trackable element thereon, the trackable element being operable for communication with a computer assisted surgery guidance system.

17. The patient specific surgical guide helmet of claim 16, wherein the trackable element is an electronic micro-electromechanical sensor (MEMS) operable to determine and provide at least orientation data of the patient specific surgical guide helmet to the computer assisted surgery guidance system.

18. A system for creating the patient specific surgical guide helmet of claim 10, the system comprising:
a surgical guide helmet generator for producing a surgical guide helmet model for positioning on the head of a patient;
a geometry identifier for identifying a helmet geometry; and
a patient specific instrument model generator receiving input from the geometry identifier to create a model of the patient-specific instrument from said helmet model, the patient specific instrument formed based on said patient specific instrument model being configured specifically for the head of the patient and adapted to be received thereon to guide said cranial surgery.

19. A method of performing a cranial surgical procedure on the head of a patient, comprising the steps of:
imaging at least a selected portion of the head of the patient and determining one or more surgical targets in at least brain tissue of the patient;
planning at least one of a trajectory, cranium entry point and end point of the surgical procedure based on the determined surgical targets;
forming a patient specific surgical guide helmet customized to the head of the specific patient based on said imaging and said planned surgical procedure, the patient specific surgical guide helmet having at least one guide element which guides a surgical implement during the cranial surgical procedure along the planned trajectory, the guide element being positioned and oriented such that an axis thereof corresponds to the planned trajectory;
securing the patient specific surgical guide helmet onto the head of the patient; and
performing the cranial surgical procedure using the guide element of the patient specific surgical guide helmet to guide the surgical implement along said axis through said planned trajectory to the determined surgical target in the brain tissue of the patient.

20. The method of claim 19, wherein the step of forming the patient specific surgical guide helmet includes:
obtaining the imagery of the head of the patient;
performing a segmentation of the imagery of said selected portion of the head of the patient;
modeling the patient specific surgical guide helmet to fit on the head of the specific patient based on said imagery; and
producing the patient specific surgical guide helmet which corresponds to the modeled patient specific surgical guide helmet.

21. The method of claim 19, wherein the step of forming the patient specific surgical guide helmet further includes forming the guide element as a guide tube having an aperture at a proximal end which provides access to the head and which defines said axis therethrough along which the surgical implement is guided, the guide tube being positioned and oriented such that the axis corresponds to the planned trajectory and the aperture defines an entry point for the surgical procedure.

22. The method of claim 19, wherein the patient specific surgical guide helmet includes at least one trackable element thereon which operable for communication with a computer assisted surgery guidance system to provide at least orientation data thereto, the method further comprising tracking at least one of position and orientation of the patient specific surgical guide helmet using the computer assisted surgery guidance system.

23. A method of performing a surgical procedure involving injecting or removing material from a target location in tissue of a patient, the method comprising the steps of:
imaging at least said tissue of the patient to generate a virtual model of the tissue, and determining one or more target locations within the virtual model of the tissue;
forming a patient specific surgical guide, customized to fit on the specific patient over a site comprising the tissue and for guiding a surgical implement used to perform the surgical procedure, by:
obtaining the imagery of the tissue of the patient;
planning at least one of a trajectory and end point of the surgical procedure based on the determined target location;
performing a segmentation of the virtual model of the tissue;
modeling the patient specific surgical guide to fit on the patient and having at least one guide element incorporated therein, the guide element including at least one aperture providing access to the tissue and defining an entry point for the surgical implement, the guide element being positioned and oriented to guide the surgical implement along the planned trajectory to the determined target location; and
creating the patient specific surgical guide which corresponds to the modeled patient specific surgical guide;
securing the patient specific surgical guide onto the patient; and
performing the surgical procedure using the patient specific surgical guide to guide the surgical implement to the determined target location in the tissue of the patient, including performing at least one of an injection of material into the target location and a removal of tissue from the target location.

24. A patient specific surgical guide for guiding a surgical procedure to be performed at a predetermined target location in tissue of a patient, comprising:
a patient-specific base portion customized in size and shape to fit directly onto a body part the specific patient, over a site comprising said tissue; and
at least one guide element integrated into the base portion, the guide element being positioned and oriented to guide a surgical implement along a planned trajectory to reach the predetermined target location within the tissue, the guide element including at least one aperture providing access to the body part and defining an entry point for the surgical procedure, and the guide element including a guide tube extending outwardly from the aperture and defining an axis centrally therein which extends through the guide tube in alignment with the aperture, the axis corresponding to the planned trajectory.
